(19) United States
(12) Patent Application Publication
(10) Pub. No.: US 2022/0257202 A1
(43) Pub. Date: Aug. 18, 2022

(54) FAIL SAFE RADIATION CONCEALMENT MECHANISM

(71) Applicant: CHECK-CAP LTD., lsfiya (IL)

(72) Inventors: Yoav KIMCHY, Haifa (IL); Elik CHEN, Naharia (IL); Avner ELGALI, Zur-lgeal Gachav-Yair (IL)

(21) Appl. No.: 17/627,816

(22) PCT Filed: Aug. 23, 2020
(86) PCT No.: PCT/IL20/50920

§ 371 Date: Jan. 18, 2022

Related U.S. Application Data
(60) Provisional application No. 62/913,204, filed on Oct. 10, 2019.

(51) Int. Cl. A61B 6/10; A61B 6/06; A61B 6/00

(52) U.S. Cl. CPC A61B 6/107; A61B 6/56; A61B 6/4057; A61B 6/06

(57) ABSTRACT

An imaging capsule including, a radiation source, a collimator that blocks the emission of radiation from the radiation source except through one or more output columns, a shutter comprising one or more openings; wherein the shutter is rotatably coupled to the collimator to enable selecting at least two states; a closed state in which the shutter blocks the emission of radiation from the radiation source, and an open state in which the shutter does not block the emission of radiation, a motor configured to rotate the collimator and select the state of the shutter, a main power source configure to power the motor, a circuit that monitors a status of the main power source and instructs the motor to place the shutter in the closed state if power in the main power source is below a threshold value.

### RELATED APPLICATIONS

The present application claims priority from U.S. Provisional application No. 62/913,204 filed on Oct. 10, 2019, the disclosure of which is incorporated herein by reference.

### FIELD OF THE INVENTION

The present invention relates generally to limiting exposure of a patient to radiation and more specifically to a fail safe radiation concealment mechanism in an imaging capsule that is swallowed by a patient to examine the patient's gastrointestinal tract.

### BACKGROUND OF THE INVENTION

One method for examining the gastrointestinal tract for the existence of polyps and other clinically relevant features that may indicate regarding the potential of cancer is performed by swallowing an imaging capsule that will travel through the tract and view the patient's situation. In a typical case the trip can take between 24-48 hours after, which the imaging capsule exits in the patient's feces. Typically the patient swallows a contrast agent to enhance the imaging ability of the imaging capsule. Then the patient swallows the imaging capsule to examine the gastrointestinal tract while flowing through the contrast agent. The imaging capsule typically includes a radiation source, for example including a radioisotope that emits X-rays or Gamma rays. The radiation is typically collimated to allow it to be controllably directed toward a specific area during the imaging process. In an exemplary case the imaging capsule is designed to detect particles from X-ray fluorescence and/or Compton back-scattering responsive to the radiation and transmit measurements (e.g. a count rate) to an external analysis device, for example a computer or other dedicated instruments.

In a typical implementation a radio-opaque contrast agent is used so that a position with a polyp will have less contrast agent and will measure a larger back-scattering count. Alternatively, other methods may be used to image the gastrointestinal tract.

U.S. Pat. No. 7,787,926 to Kimchy the disclosure of which is incorporated herein by reference, describes details related to the manufacture and use of such an imaging capsule.

Use of an imaging capsule exposes the user to radiation, which may be potentially harmful. Accordingly, it is of interest to limit the user's exposure to radiation when not necessary, for example while the imaging capsule is located in positions that do not need to be measured. Typically, the imaging capsule may be designed with shutters that can be instructed to block the exit of radiation when not needed. However, there still exists the hazard that in case of malfunction of the imaging capsule, for example in case of a power failure radiation may be emitted without constraint.

It is thus desirable to design a fail safe radiation blocking mechanism that automatically blocks the emission of radiation and only allows radiation to be emitted if power is available and the device provides an instruction to allow radiation to be emitted.

### SUMMARY OF THE INVENTION

An aspect of an embodiment of the invention, relates to an imaging capsule for scanning inside a living body, with a fail-safe radiation mechanism that prevents uncontrolled release of radiation from the imaging capsule, for example due to a power failure while taking images. When the power fails the imaging capsule cannot restore the position of a shutter to block the release of radiation from the imaging capsule due to lack of power and the user risks being over exposed. Therefore the imaging capsule includes an auxiliary power source that is configured to use the power to move the shutter back to block radiation in case of failure of the main power source.

There is thus provided according to an exemplary embodiment of the disclosure, an imaging capsule with a fail-safe radiation mechanism, comprising:

a radiation source;

a collimator that blocks the emission of radiation from the radiation source except through one or more output columns;

a shutter rotatably coupled to the collimator enabled to select at least two states; a closed state in which the shutter blocks the emission of radiation from the radiation source, and an open state in which the shutter does not block the emission of radiation;

a motor configured to rotate the collimator and select the state of the shutter;

a main power source configure to power the motor;

a circuit that monitors a status of the main power source and instructs the motor to place the shutter in the closed state if power in the main power source is below a threshold value.

In an exemplary embodiment of the disclosure, the shutter comprises one or more openings; and wherein in the closed state the openings of the shutter do not coincide with the output columns so that the shutter blocks the emission of radiation from the radiation source, and in the open state the openings of the shutter coincide with the output columns and do not block the emission of radiation. Optionally, in the open state the shutter and the collimator are held together by one or more magnets installed in the shutter and collimator. In an exemplary embodiment of the disclosure, the magnets of the shutter and collimator are separated the shutter blocks radiation emitted from the collimator. Optionally, in the open state the shutter and the collimator are held together by one or more magnets installed in the shutter and the collimator includes a ferromagnetic material to be attracted to the shutter or vice versa.

In an exemplary embodiment of the disclosure, when the magnets of the shutter or collimator are separated the shutter blocks radiation emitted from the collimator. Optionally, the shutter includes an extrusion that is blocked by a stationary stopper that prevents the shutter from completing a complete rotation of 360 degrees. In an exemplary embodiment of the disclosure, the stopper is configured to move the shutter relative to the collimator and change the state of the imaging capsule from the open state to the closed state and vice versa.

In an exemplary embodiment of the disclosure, the imaging capsule comprises an auxiliary power source that provides power to place the shutter in the closed state. Optionally, the auxiliary power source is initially charged from the main power source.

There is further provided according to an exemplary embodiment of the disclosure, a method of providing fail safe radiation in an imaging capsule, comprising:

installing a radiation source within a collimator that blocks the emission of radiation from the radiation source except through one or more output columns;

rotatably coupling a shutter the collimator to enable selecting at least two states; a closed state in which the shutter blocks the emission of radiation from the radiation source, and an open state in which the shutter does not block the emission of radiation;

attaching a motor configured to rotate the collimator and select the state of the shutter; wherein the motor is powered by a main power source;

monitoring a status of the main power source with a circuit;

and

if the power in the main power source is below a threshold value instructing the motor to place the shutter in the closed state.

### BRIEF DESCRIPTION OF THE DRAWINGS

The present invention will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a cross sectional view of an imaging capsule with a failsafe radiation emission system, according to an exemplary embodiment of the invention;

FIG. 2A is a schematic illustration of a shutter and collimator in an open state enabling the release of radiation to take images of a gastrointestinal tract of a user, according to an exemplary embodiment of the disclosure;

FIG. 2B is a schematic illustration of a shutter with an extrusion abutting a stopper to rotate the shutter relative to the collimator from an open state to a closed state, according to an exemplary embodiment of the disclosure;

FIG. 2C is a schematic illustration of a shutter and collimator in a closed state prevent the release of radiation from an imaging capsule, according to an exemplary embodiment of the disclosure:

FIG. 2D is a schematic illustration of a transparent view of a shutter rotatably coupled to a collimator, according to an exemplary embodiment of the disclosure;

and

FIG. 3 is a schematic illustration of a perspective internal view of an imaging capsule, according to an exemplary embodiment of the invention.

### DETAILED DESCRIPTION

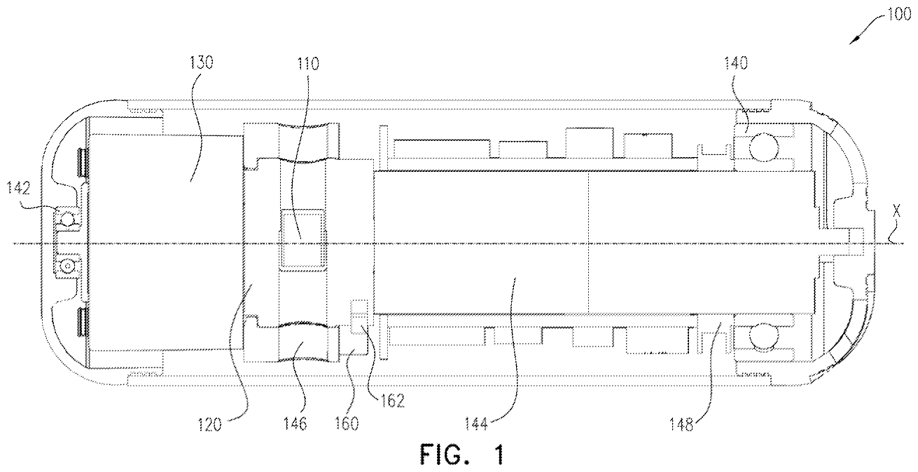

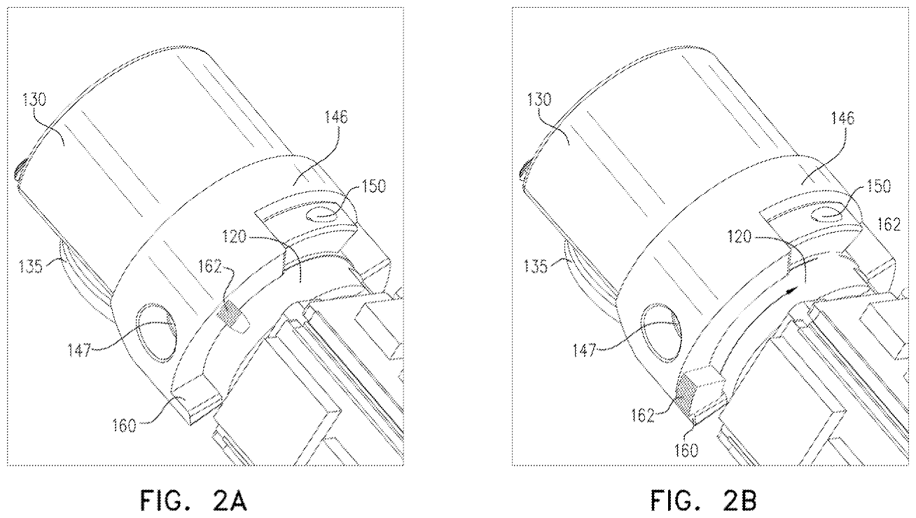

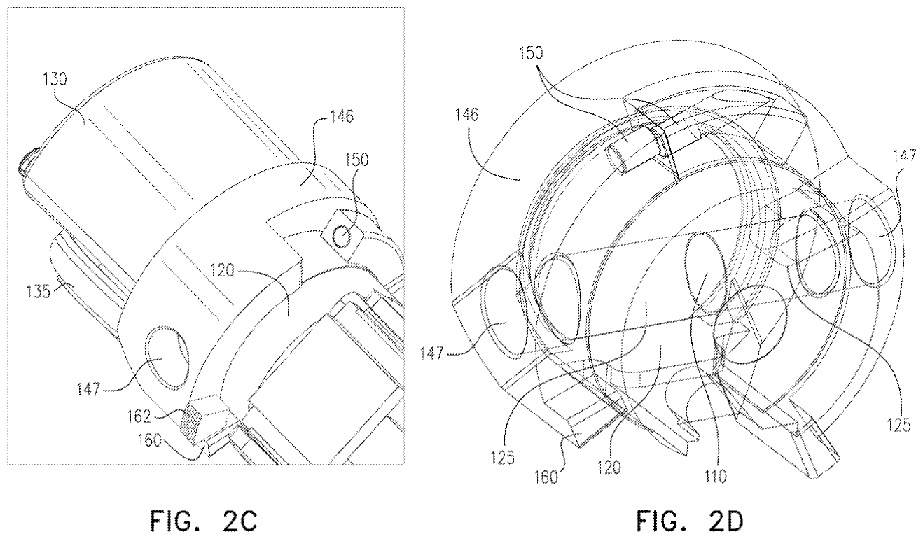

FIG. 1 is a schematic illustration of a cross sectional view of an imaging capsule 100 with a failsafe radiation emission system 100, according to an exemplary embodiment of the invention. In an exemplary embodiment of the invention, a patient swallows a contrast agent which mixes with the content of their gastrointestinal tract to increase the accuracy of the radiation measurements. Then the patient swallows imaging capsule 100 to examine the gastrointestinal tract as the imaging capsule 100 proceeds through the gastrointestinal tract. In an exemplary embodiment of the invention, imaging capsule 100 includes a collimator 120 with output columns 125 for directing radiation (see FIG. 2D), and a shutter 146 rotatably coupled to the outer circumference of the collimator 120. The shutter 146 is designed to selectively enable or block radiation from being emitted from a radiation source 110 through collimator 120. Initially radiation is blocked and upon receiving instructions to release radiation the shutter 146 is turned relative to the collimator 120 to release radiation. The release of radiation allows the imaging capsule 100 to take images of the environment surrounding the imaging capsule 100. Optionally, selection of all the different modes of operation (e.g enabling radiation/blocking radiation) is performed by rotating the collimator 120 with a motor 144.

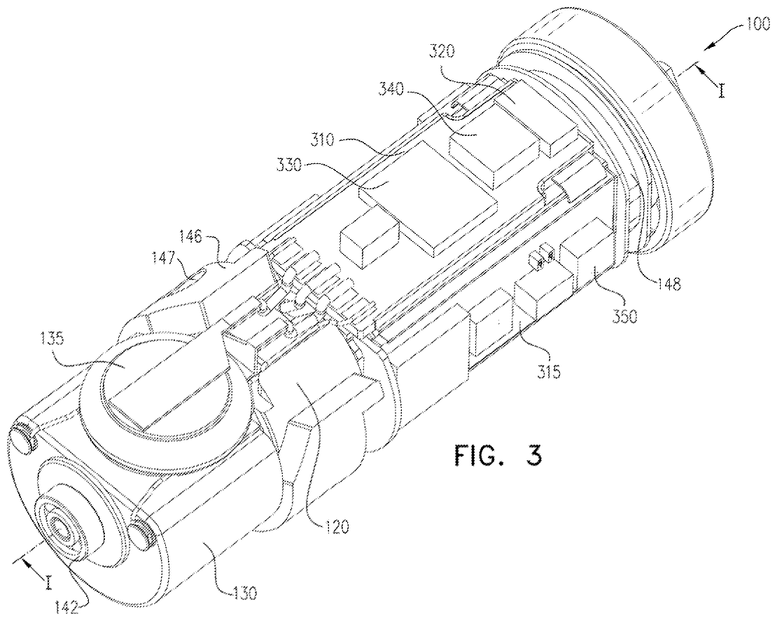

In an exemplary embodiment of the invention, imaging capsule 100 applies power from a main power source 130 (e.g. a battery) to turn collimator 120 relative to shutter 146 to select an open state that enables the emission of radiation and to select a closed state that blocks the emission of radiation. Optionally, imaging capsule 100 includes an auxiliary power source 135 (see FIG. 3), which applies power upon failure or depletion of the main power source 130 to return the shutter 146 back to its original position relative to collimator 120 to block the emission of radiation. In an exemplary embodiment of the disclosure, the auxiliary power source 135 is a battery, a capacitor or an inductor. Optionally, the auxiliary power source 135 may be initially charged from the main power source 130 and configured to preserve the charge after the main power source 130 is depleted. Alternatively, imaging capsule 100 may include two separate batteries. Optionally, one may be larger to power the imaging capsule and the other may be smaller with enough power just to position the shutter 146 to block radiation.

In an exemplary embodiment of the disclosure, imaging capsule 100 includes a front bearing 140 and a rear bearing 142 that are configured to support motor 144 that rotates around an elongated axis (X) of the imaging capsule, for example under the influence of an electronic coil 148. In an exemplary embodiment of the disclosure, when imaging the gastrointestinal tract the motor 144 is generally programed to rotate the collimator 120 back and forth, for example about 270 degrees with an overlap of about 110 degrees between two scanning sectors (see FIG. 2D—scanning from two sides of the imaging capsule to cover the entire inner contour of the gastrointestinal tract).

Optionally, the motor 144 rotates collimator 120, shutter 146 and radiation source 110 and is also configured to cause the shutter 146 to be in the open state or closed state. When shutter 146 is in the open state, collimator 120 releases radiation through output columns 125 and openings 147 on the shutter 146, which coincide with the output columns 125. When shutter 146 is in a closed state, shutter 146 blocks the output columns 125 and prevents radiation from exiting the collimator 120.

FIG. 2A shows shutter 146 in the opened state. In an exemplary embodiment of the disclosure, friction of the motor and any gears implemented within the motor prevent the shutter 146 and collimator 120 from slipping from one state to another. Alternatively or additionally, shutter 146 and/or collimator 120 include magnets 150 to secure shutter 146 relative to collimator 120 to keep the imaging capsule in the open state and prevent them from accidently swapping states. In some embodiments of the disclosure, both the shutter 146 and the collimator 120 have magnets attracting each other. Alternatively, only the shutter 146 has one or more magnets installed and the collimator 120 has installed a ferromagnetic metal that is attracted to the magnet, or vice versa.

In an exemplary embodiment of the disclosure, shutter 146 includes an extrusion 160 and the imaging capsule 100 also includes a stopper 162 to match the extrusion. The stopper 162 is stationary in the path of the extrusion 160 (e.g. on an inside wall of an encasement 105 surrounding the imaging capsule). Optionally, all the components shown in FIG. 1 except for stopper 162 rotate around axis X, with the help of front bearing 140 and rear bearing 142.

When imaging the gastrointestinal tract the collimator 120 and shutter 146 rotate together back and forth imaging an entire circumference around the collimator 120. When imaging capsule 100 is instructed to block imaging, the motor 144 continues the rotation (e.g. clockwise) until the extrusion 160 meets the stopper 162 (e.g. as shown in FIG. 2B) then instead of turning back the other way to continue scanning, the motor 144 continues to turn the collimator 120 and the stopper 162 pushes the extrusion 160 of the shutter 146 separating the magnets 150 of the shutter 146 and the collimator 120, for example as shown in FIG. 2C (the closed state). In this state (the closed state) the shutter 146 blocks the output columns 125 of the collimator 120 and prevents the release of radiation from radiation source 110. Optionally, by rotating the collimator 120 in the opposite direction the shutter 146 can be turned back to the opened state.

In an exemplary embodiment of the disclosure, stopper 162 and extrusion 160 prevent the shutter 146 from completing rotation of 360 degrees. When rotating the collimator 120 in either direction (clockwise or counterclockwise) the shutter 146 is stopped by the stopper 162 right before finishing the complete rotation.

FIG. 3 is a schematic illustration of a perspective internal view of an imaging capsule 100, according to an exemplary embodiment of the invention. Optionally, imaging capsule 100 includes a circuit 310 with various components, for example a processor 320, a real time clock 340, sensors 330, a communication controller 350 and/or other electronic components to control functionality of the imaging capsule.

In an exemplary embodiment of the disclosure, if power source 130 fails or falls below a threshold value, components of the electronic circuit 310 activate the auxiliary power source 135 to provide power to return the shutter 146 relative to the collimator 120 to the closed state, blocking the release of radiation. Optionally, in case of a software failure or if radiation is released for a time interval greater than a preselected value the electronic circuit 310 will instruct the motor to return shutter 146 to block the release of radiation.

In an exemplary embodiment of the disclosure, a separate electronic circuit 315 monitors the main power source 130 and if the voltage drops below a threshold value, the circuit 315 overrides electronic circuit 310 and closes the shutter 146 by turning the motor in a required direction to close the shutter 146 using the remaining power in the main power source 130.

Alternatively, a separate electronic circuit 315 monitors the main power source 130 and activates the auxiliary power source 135 in case of failure with the main power source 130. This enhances reliability of the imaging capsule since an independent electronic circuit 315 and independent power source 135 are in charge of blocking radiation in case of a power failure.

In an exemplary embodiment of the disclosure, stopper 162 extends from the circuit 310, circuit 315 or other stationary elements in the imaging capsule 100 so that the stopper 162 is in a fixed position to prevent extension 160 from forming complete rotational cycles.

In an exemplary embodiment of the disclosure, the motor 144 is activated and/or deactivated responsive to measurements from the sensors 330. Alternatively or additionally, the motor 144 may be controlled responsive to commands received from an external controller via communication controller 350.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the invention. Further combinations of the above features are also considered to be within the scope of some embodiments of the invention.

It will be appreciated by persons skilled in the art that the present invention is not limited to what has been particularly shown and described hereinabove. Rather the scope of the present invention is defined only by the claims, which follow.

We claim:

1. An imaging capsule with a fail-safe radiation mechanism, comprising:
a radiation source;
a collimator that blocks the emission of radiation from the radiation source except through one or more output columns;
a shutter rotatably coupled to the collimator enabled to select at least two states; a closed state in which the shutter blocks the emission of radiation from the radiation source, and an open state in which the shutter does not block the emission of radiation;
a motor configured to rotate the collimator and select the state of the shutter;
a main power source configure to power the motor;
a circuit that monitors a status of the main power source and instructs the motor to place the shutter in the closed state if power in the main power source is below a threshold value.

2. The imaging capsule according to claim 1, wherein the shutter comprises one or more openings; and wherein in the closed state the openings of the shutter do not coincide with the output columns so that the shutter blocks the emission of radiation from the radiation source, and in the open state the openings of the shutter coincide with the output columns and do not block the emission of radiation.

3. The imaging capsule according to claim 1, wherein in the open state the shutter and the collimator are held together by one or more magnets installed in the shutter and collimator.

4. The imaging capsule according to claim 3, wherein when the magnets of the shutter and collimator are separated the shutter blocks radiation emitted from the collimator.

5. The imaging capsule according to claim 1, wherein in the open state the shutter and the collimator are held together by one or more magnets installed in the shutter and the collimator includes a ferromagnetic material to be attracted to the shutter or vice versa.

6. The imaging capsule according to claim 5, wherein when the magnets of the shutter or collimator are separated the shutter blocks radiation emitted from the collimator.

7. The imaging capsule of according to claim 1, wherein the shutter includes an extrusion that is blocked by a stationary stopper that prevents the shutter from completing a complete rotation of 360 degrees.

8. The imaging capsule according to claim 7, wherein the stopper is configured to move the shutter relative to the collimator and change the state of the imaging capsule from the open state to the closed state and vice versa.

9. The imaging capsule according to claim 1, wherein the imaging capsule comprises an auxiliary power source that provides power to place the shutter in the closed state.

10. The imaging capsule according to claim 9, wherein the auxiliary power source is initially charged from the main power source.

11. A method of providing fail safe radiation in an imaging capsule, comprising:
installing a radiation source within a collimator that blocks the emission of radiation from the radiation source except through one or more output columns;
rotatably coupling a shutter the collimator to enable selecting at least two states; a closed state in which the shutter blocks the emission of radiation from the radiation source, and an open state in which the shutter does not block the emission of radiation;
attaching a motor configured to rotate the collimator and select the state of the shutter; wherein the motor is powered by a main power source;
monitoring a status of the main power source with a circuit;
and
if the power in the main power source is below a threshold value instructing the motor to place the shutter in the closed state.

12. The method according to claim 1, wherein the shutter comprises one or more openings; and wherein in the closed state the openings of the shutter do not coincide with the output columns so that the shutter blocks the emission of radiation from the radiation source, and in the open state the openings of the shutter coincide with the output columns and do not block the emission of radiation;

13. The method according to claim 11, wherein in the open state the shutter and the collimator are held together by one or more magnets installed in the shutter and collimator.

14. The method according to claim 13, wherein when the magnets of the shutter and collimator are separated the shutter blocks radiation emitted from the collimator.

15. The method according to claim 11, wherein in the open state the shutter and the collimator are held together by one or more magnets installed in the shutter and the collimator includes a ferromagnetic material to be attracted to the shutter or vice versa.

16. The method according to claim 15, wherein when the magnets of the shutter or collimator are separated the shutter blocks radiation emitted from the collimator.

17. The method according to claim 11, wherein the shutter includes an extrusion that is blocked by a stationary stopper that prevents the shutter from completing a complete rotation of 36 degrees.

18. The method according to claim 17, wherein the stopper is configured to move the shutter relative to the collimator and change the state of the imaging capsule from the open state to the closed state and vice versa.

19. The method according to claim 11, wherein the imaging capsule comprises an auxiliary power source that provides power to place the shutter in the closed state.

20. The method according to claim 19, wherein the auxiliary power source is initially charged from the main power source.
